(19) United States
(12) Patent Application Publication
(10) Pub. No.: US 2019/0070043 A1
(43) Pub. Date: Mar. 7, 2019

(54) Adaptable and Dynamic Incontinence Wetness Sensor

(71) Applicant: Daniel Ross Collette, Albuquerque, NM (US)

(72) Inventor: Daniel Ross Collette, Albuquerque, NM (US)

(21) Appl. No.: 15/694,768

(22) Filed: Sep. 2, 2017

(51) Int. Cl. A61F 13/42; G08B 21/20; G01N 27/04

(52) U.S. Cl. CPC A61F 13/42; G08B 21/20; A61F 5/48; A61F 2013/427; G01N 27/048

(57) ABSTRACT

This non-provisional patent filing is a follow-on to U.S. Pat. No. 8,421,636 B2 issued Apr. 16, 2013 and is to document the development work done between the original patent filing and continued product research and development. With increased interest in the realm of incontinence wetness sensing in adults and children within a wide variety of conditions, there is always at the core of the sensing, the need for accuracy. Without accurately predicting a wet event, a system rapidly becomes useless and is abandoned. Ongoing development and implementation of incontinence wetness sensing, in a wide variety of venues has identified significant limitations in current incontinence monitoring systems. In theater test and development data has shown that with any incontinence wetness sensing that key measurement parameters vary much more widely than previously predicted. The measurement accuracy of these parameters can significantly affect the reliability of wetness sensing second, third and fourth wet events of an incontinent product. This patent claims the implementation of a wide dynamic range sensing system that utilizes digital variable resistance, capacitance or other measuring technique. This new sensing method, uniquely adapts to the incontinent product environment significantly improving the sensing range and tailored response. The use of these configurable elements allows for modification in real time by microcontroller or other controlling device of the reference in wetness sensing applications enabling this in system dynamic reconfigurable capability.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM (EFS-WEB)

Not Applicable

### STATEMENT REGARDING PRIOR DISCLOSURES BY THE INVENTOR OR A JOINT INVENTOR

Not Applicable

### BACKGROUND OF THE INVENTION

Systems for sensing wetness are well documented back to the 1950's where Sears and Roebuck sold a “Wee-Alert” Bed Wetting Alarm. This fact limits or completely eliminates aspects of many patents applications and in fact patents issued since that early 1950's date of sale for a wetness sensor with notification. This puts a real burden of proof on any new patents in the field of wetness sensing to demonstrate or show that the claims made are indeed novel and not just an iteration of prior art that would be obvious to one familiar or skilled in the subject material.

There is also a distinct difference between an wetness notification system and a wetness sensing system. A notification system, merely would acquire data from a sensor which is developed and designed to detect wetness and process that wet notification to a caregiver or user. On the other hand, a wetness sensor would be the mechanics and methodology of properly determining a wet event and feeding that notification into a wetness sensing system for processing. These two concepts are distinctly different and must be treated as such. A couple of examples of the complexity of sensing wetness determined during over 20 years of development would demonstrate this. In early trials of a wetness sensing system prior to the filing of the Collette et al [US 2005/0033250] at Kimberly Clark, failures to properly sense wetness in non-woven disposable diapers led to the discovery of the impact of non-woven material in the generation of static in the diaper leading to significant failures in current wet sensing capabilities. That led to the development and patent of algorithms and methods to remove static events as triggers for wetness sensing. Another example of the criticality of sensors in wetness systems was one that led to the filing of this paten application. While implementing a resident monitoring system for nursing homes, assisted living centers and hospice, a higher than expected ratio of false alarms was detected in the hospice application. Analysis of the data showed that as the resident became increasingly ill, the decline in kidney function led to a significant increase in mineral content excreted by the kidneys. This resulted in the sensor as designed being unable to correctly measure an incontinent event even with designed in level adjustments. These factors, even to one experienced in the art, would not lead to the development and patentability of critical sensor design modifications, without significant investment of time and material resources, which are what patents are intended to protect.

### BRIEF SUMMARY OF THE INVENTION

In all prior art, the sensing of wetness, and that includes wetness sensing clear back to the 1950's Sears and Roebuck, “Wee-Alert” alarm system, has been done by threshold levels. A sensor sends a resistance, capacitance or other measure through an analog comparator which has a set of threshold(s) which then trigger a wet notification. Most often, these threshold levels are changed by manual input and require changes to board configurations or software that changes the comparator to a different resistor via a mux. Digital resistors became available around 2004. Even through they have been available, there have been no wetness sensors to date that make use of that technology in their systems. This patent is that the sensor is not only adjustable via the digital resistor but that adjustment is achieved automatically by the sensor itself.

### BRIEF DESCRIPTION OF THE SEVERAL VIEWS OF THE DRAWING(S)

Not applicable (See the Detailed Description of the Invention)

### DETAILED DESCRIPTION OF THE INVENTION

The current versions of wetness sensors as described in U.S. Pat. No. 8,421,636 B2 issued Apr. 16, 2013 do a very good job of detecting, without false alarms, the incontinent product users initial wet event in a dry incontinent product. The ability to detect subsequent wet events and diaper saturation is a more challenging problem due to the wide range of user urine salinity which is the main factor in the urine resistance/conductance level.

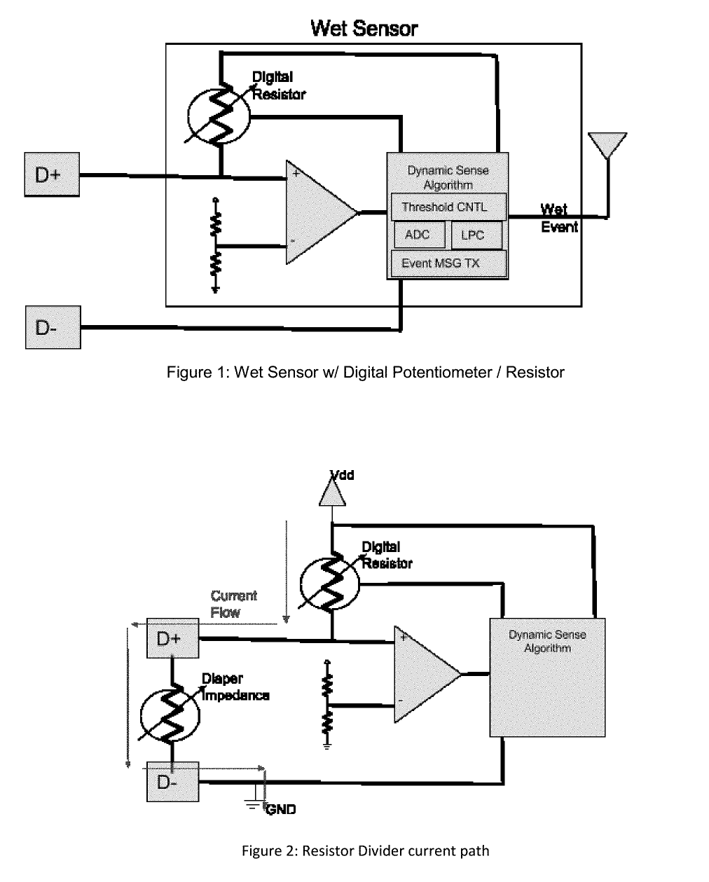

Following the initial wetness event, the ability to dynamically report the exact resistance value of the garment after the wet event and then set wetness sensing unit's new threshold resistance level based on this user's urine resistance enables a new class of wetness sensing capabilities. It will enable the sensor to better track a user's current wetness and detect subsequent wet events temporally correlated to the user's current hydration and health. Providing this data back to the cloud also enables long term tracking of a user's urine salinity which could potentially enable the detection of a change in the user's health and potentially predict and or catch an issue well before it is evident by other means.
See FIG. 1: Wet Sensor w/Digital Potentiometer/Resistor

The temporal correlation of a user's urine resistance should be very close from wet event to wet event but rate of change resistance of the users urine will be measurable based on temperature, i.e. lower resistance immediately after incontinent event and it will reduce as urine cools within the incontinent product. Utilizing the dynamic tracking function of the new wetness sensor should enable the ability to detect on new wet events and also track overall diaper saturation based on time since first event, subsequent events and delta change in resistance.

The addition of a digital potentiometer to the wetness sensor enables the ability to change comparator thresholds based on in-system and real time measurable parameters but still enable the ultra low power operation of comparator based sensing.

With the dynamic configuration of the new wetness sensor married with a system level enabled tracking and reporting system the wetness detecting system now has the ability to more intelligently report initial and subsequent incontinent events, over all diaper saturation and potentially the incontinent users heath (as it pertains to the urinary tract) Adding additional measurement systems, such as temperature, capacitance, ph could help in the detection determination of a secondary wetness events but will ultimately drive up the complexity and cost of the diaper and sensor and making it less affordable. These additional sensing elements will be considered more in the future.

The following section will cover the implementation of this new technology into the System.

Wet Sensor Dynamic Algorithm Description

The algorithm updates to support the new dynamic capability of the wet sensor includes updates to the Wet Sense Unit firmware and the System level monitor (local webpage).

A high level overview of the new algorithm flow for the Wet Sense Unit:
1. Wet Sense Unit attached to new/dry incontinent garment
2. Wet Sense Unit initialized sensing threshold for a dry incontinent garment (human interaction or autonomously), checks to ensure garment is dry.
3. Wet Sense Unit triggers on the first wet/incontinent event
4. Wet Sense Unit determines what the resistance of the garment is post the event.
5. Wet Sense Unit reports to system the resistance level the diaper triggered on and the new resistance value of the garment
6. Wet Sense Unit delays TBD minutes to allow the diaper to absorb the wet event.
7. Wet Sense Unit sets a new and sensing threshold that is an offset from the resistance value that triggered the previous to provide margin/hysteresis against false alarms in an already wet incontinent product.
8. Wet Sense Unit based on the new resistance threshold value and to preserve battery life the unit will set the trigger mode to edge triggered (>10 KOhm) or periodic level sense (<10 KOhm)
9. Wet Sense unit enables wet sense capability with new threshold set to monitor for the next incontinent/wet event.

Detailed updates to enable dynamic sense capability to the wet sense algorithm are captured below.

Overview

In prior version of the Wetness sensor the threshold values of the sensor were set by discrete resistance components. This enabled very accurate initial wetness detection but for subsequent incontinent events the fixed resistance components did not allow for an adaptable and dynamic reconfiguration. The new sensor utilizes a digital potentiometer to facilitate this new adaptable and dynamic reconfiguration.

The analog devices AD5165 is a digital potentiometer and has been integrated into the design and will enable this new function but any digital potentiometer would suffice.

The digital potentiometer is 100K Ohm end to end, between port A and B. The sensing will take place off of port W which we can configure between 100K to 0 Ohms. There are 256 resistance steps between 100K to 0 Ohm, this is represented by a 8 bit digital value, resulting in a resolution of 390 Ohms per step.

This enables the sensor to determine urine resistances of less than or equal to 50K Ohm at 390 Ohm of resolution per step (Current=7 mA per step, Conductance 2.5 mS per step) This results in a dynamic resistance command word format of: bXXXX_XXXX, where the most significant bit X represents a configurable value of 1 or 0.

This results in a control dynamic control range of:

B1111_1111 (^{˜}100 kohm)) to b000_0000 (^{˜}0 Ohm)

Initialization:

The initialization of the algorithm does not change between this version and past versions.

There are three types of initialization events: power on reset, magnet/proximity swipe connection check or the periodic system connection check.

Initialization: Power on Reset:

The power on reset initialization is executed when the battery is applied. The digital resistor is set to 50 KOhms initializing the sensing threshold to a dry garment.

Initialization: Magnet/Proximity Swipe Connection Test

During an incontinent product change the caregiver will swipe the Wet Sense unit with a magnet. This will trigger a diaper connection test event. The connection event currently checks to see if the diaper is connected correctly to the garment.

As part of this algorithm update the initial threshold/resistance value will also be dynamically set.

Magnet/Proximity Swipe Connection Test Algorithm flow:
1. Wet Sense Unit senses a magnet swipe
2. Disables the wet sense interrupt.
3. Checks Diaper connection, sets pass/fail flag for D+ and D−
4. Sets the digital resistor to 50 KOhm
5. Verifies that current threshold value does not trip the comparator or that voltage is above threshold voltage.
   a. If not tripped continue to step 6
   b. If tripped, set threshold value to one half less than current value recheck threshold.
      i. Continue until non trip threshold is found, comparator is no longer tripped.
      ii. Once found add % of the current value until comparator trips
      iii. Reduce by 1 KOhm steps until comparator is no longer tripped.
      iv. This is the current diaper resistance level.
6. Send Diaper connection test message to the system with the current diaper resistance level (50 KOhm for dry diaper).
   a. “O” Message for passed connection test
   b. “E” Message for failed connection test
   c. Diaper resistance level does not impact pass/fail message determination
   d. Message format defined below.
7. If diaper resistance level is not 50 KOhm, subtract TBD KOhm from the current resistance level for new threshold level
8. If resistance threshold level is >10 KOhm enable edge detection mode, if <10 KOhms enable periodic level sense mode.

The Message format will be updated to include the current resistance setting/threshold.

TABLE
Old (WAS) Magnet Swipe Diaper Check Message Format

| | Byte | | | | | | |
|---|---|---|---|---|---|---|---|
| | Byte 0 | Byte 1 | Byte 2-9 | Byte 10 | Byte 10-11 | Byte 12 | Byte 13-14 |
| Name | Diaper | delimiter | Sensor | delimiter | Battery | delimiter | Connection |
| | Connect | | MAC ID | | Voltage | | Status |
| | Pass/Fail | | | | | | |
| Value | Asci “O” | Asci ‘\hat{ }’ | 8xAsci | Asci ‘\hat{ }’ | ADCH(7:0) ++ | Asci ‘\hat{ }’ | Asci ‘++’, ‘+−’, |
| | or “E” | | Char | | ADCL(7:2) | | ‘−+, or ‘−−’ |

TABLE
New (IS) Diaper Check Message Format

| | Byte | | | | | | | | |
|---|---|---|---|---|---|---|---|---|---|
| | Byte 0 | Byte 1 | Byte 2-9 | Byte 10 | Byte 10-11 | Byte 12 | Byte 13-14 | Byte 15 | Byte 16-19 |
| Name | Diaper | delimiter | Sensor | delimiter | Battery | delimiter | Connection | delimiter | Threshold And |
| | Connect | | MAC ID | | Voltage | | Status | | Setpoint |
| | Pass/Fail | | | | | | | | |
| Value | Asci “O” | Asci ‘\hat{ }’ | 8xAsci | Asci ‘\hat{ }’ | ADCH(7:0) ++ | Asci ‘\hat{ }’ | Asci ‘++’, ‘+−”, | Asci ‘\hat{ }’ | Threshold and |
| | or “E” | | Char | | ADCL(7:2) | | ‘−+, or ‘−−’ | | Set Point |
| | | | | | | | | | Resistance |

Connection Status:
0=D+ and D− not connected,
1=D+ not connected and D− connected
2=D+ connected and D− not connected
3=D+ and D− connected,

At the system level, if the value is 50K Ohm it signifies the garment is dry. If the resistance value is less than 50K Ohm, it may indicated that the garment is already wet. This information will be used by the system to determine notification type.

Initialization: Periodic Connection Test

The periodic connection test executes every TBD minutes to check that the diaper is still connected correct and to check the wetness of the product and set the sensor back to 50 KOhm if garment was changed but not swiped.

This algorithm is similar to the magnet swipe connection test algorithm except the only check performed against the diaper resistance is if dry diaper threshold of 50 KOhm is valid, if it is not the threshold is set back to the current threshold

Magnet Swipe Connection Test Algorithm flow:
1. Wet Sense Unit senses a magnet swipe
2. Disables the wet sense interrupt.
3. Checks Diaper connection, sets pass/fail flag for D+ and D−
4. Stores current digital resistor value
5. Sets the digital resistor to 50 KOhm
6. Verifies that current threshold value does not trip the comparator or that voltage is above threshold voltage.
   a. If not tripped continue to step 6
   b. If tripped, set threshold value back to current value.
7. Send Diaper connection test message to the system with the current diaper resistance level
   a. “T” Message for passed connection test
   b. “L” Message for failed connection test
   c. Diaper resistance level does not impact pass/fail message determination
   d. Message format defined below.
8. If resistance threshold level is >10 KOhm enable edge detection mode, if <10 KOhms enable periodic level sense mode.

The Message format will be updated to include the current resistance setting/threshold.

TABLE
Old (WAS) Periodic Connection Check Message Format

| | Byte | | | | | | |
|---|---|---|---|---|---|---|---|
| | Byte 0 | Byte 1 | Byte 2-9 | Byte 10 | Byte 10-11 | Byte 12 | Byte 13-14 |
| Name | Diaper | delimiter | Sensor | delimiter | Battery | delimiter | Connection |
| | Connect | | MAC ID | | Voltage | | Status |
| | Pass/Fail | | | | | | |
| Value | Asci “T” | Asci ‘\hat{ }’ | 8xAsci | Asci ‘\hat{ }’ | ADCH(7:0) ++ | Asci ‘\hat{ }’ | Asci ‘++’, ‘+−”, |
| | or “L” | | Char | | ADCL(7:2) | | ‘−+, or ‘−−’ |

TABLE
New (IS) Periodic Connection Check Message Format

| | Byte | | | | | | | | |
|---|---|---|---|---|---|---|---|---|---|
| | Byte 0 | Byte 1 | Byte 2-9 | Byte 10 | Byte 10-11 | Byte 12 | Byte 13-14 | Byte 15 | Byte 16-19 |
| Name | Diaper | delimiter | Sensor | delimiter | Battery | ddelimiter | Connection | delimiter | Threshold and |
| | Connect | | MAC ID | | Voltage | | Status | | Set Point |
| | Pass/Fail | | | | | | | | |
| Value | Asci “T” | Asci ‘\hat{ }’ | 8xAsci | Asci ‘\hat{ }’ | ADCH(7:0) ++ | Asci ‘\hat{ }’ | Asci ‘++’, ‘+−”, | Asci ‘\hat{ }’ | Threshold |
| | or “L” | | Char | | ADCL(7:2) | | ‘−+, or ‘−−’ | | Resistance |
| | | | | | | | | | and Set Point |

Connection Status:
0=D+ and D− not connected,
1=D+ not connected and D− connected
2=D+ connected and D− not connected
3=D+ and D− connected,

The periodic reporting the incontinent product connection status and current threshold level enables monitoring of the sensing system and incontinent product's state of health.

Monitoring Operation:

After the initialization phase of the sensor unit it will enter monitoring mode. There are two monitoring modes of the wet sensor; edge monitoring and periodic level monitoring.

The reason for having the two modes is power conservation which directly impacts battery life. The updated wet sense units ability to dynamically sense and track subsequent wet events over a very large dynamic range requires the sensor unit to configure the wet sense threshold/resistance to levels low enough to enable sense additional wet events. In older versions of the wet sense unit its dynamic range was very limited and the unit would routinely saturate after the first wet event making it unable to detect additional wet events.

The power required to monitor increases inversely to the threshold setting. As the incontinent product becomes more saturated the threshold/resistance level lowers and the power required to monitor at that new threshold goes up.

To maintain battery life if the threshold/resistance drops below TBD KOhms the unit will switch between the always on, always monitoring, edge monitoring mode to the periodic level monitoring mode.

These two monitoring modes functions and their differences are captured below.

Monitoring: Edge Monitoring

The edge monitoring mode of the wet sensor is the default mode of the sensor, it is also the historical/classical sensing mode and has been utilized since the very first versions of the wet sense model. The implementation of this mode is not changing and only and only an implementation overview will be provided in this document.

Edge monitoring mode is always on and always monitoring enabling the wet sense unit to capture wet events real time, as they are occurring.

High level flow:
1. Enable the threshold/resistor pull up voltage
   a. This enables the sensing of the incontinent product
   b. Driven from a micro controller general purpose I/O (GPIO)
2. Enable Comparator Interrupt
   a. This enables the monitoring of threshold set point
3. Monitor for a wet event
   a. The micro controller is put into a sleep mode
   b. If the diaper impedance drops lower than the threshold set point the comparator will trip.
   c. This change in state of the comparator triggers an interrupt inside the wet sense unit micro controller, waking it up from a lower power state to capture the wet event.
   d. If wet event goto Wet Event processing
   e. If not wet, continue monitoring

The power consumed by the comparator and micro controller while in the sleep mode is very low.

The power utilization increase comes in the form of the resistor divider created by the pull up threshold resistor and the pull down diaper impedance. As the diaper impedance/resistance drops so does the pullup threshold resistance increasing the amount of current that can flow between VDD and Ground. FIG. 2 (Resistor Divider Current Path) illustrates this current path.

Monitoring: Periodic Level Monitoring

When both the Diaper Impedance and threshold/resistance (Digital Resistor in figure above) drop below TBD K Ohms the sensor will need to cut off this constant current supply and enter its periodic level monitoring mode.

The periodic level monitoring mode has two phases:
1. Sleep Phase:
   a. The threshold monitoring circuit is disabled and the unit is consuming very little power
   b. The circuit is in this phase a majority of the time.
2. Level Monitoring Phase:
   a. Level threshold monitoring is enabled and a level wet check is done
   b. The power configuration for this phase is much higher but it is only in this phase

When in this mode the sleep phase and the level monitoring phase are repeated over and over at a periodic interval, hence the name periodic level monitoring.

The configuration flow of the periodic level monitoring mode is:
1. Disable the comparator interrupt
   a. This disables edge monitoring mode by disabling wet event interrupt routine that runs when the comparator trips.
   b. The comparator is still working but needs to be checked manually by the processor
2. Disable the dynamic resistor pull up voltage
   a. This cuts off the current flow path shown in the figure above
3. Set a sleep timer for TBD minutes
   a. This puts the sensor into a very low power mode for TBD minutes
   b. During this time it is not actively sensing for a wet event
4. The sensor wakes up after TBD minutes
5. Enable the dynamic resistor pull up voltage
6. Check the comparator level, tripped versus not tripped
   a. Not tripped:
      i. Goto step 1
      ii. This encompasses the periodic monitoring loop
   b. Tripped:
      i. Goto Wet Event Processing

Wet Event Processing

If either the edge monitoring and periodic level monitoring modes trigger a wet event both will enter the wet event processing function to transmit a wet event message and set the unit back up for additional monitoring.

The Wet Event Processing Algorithm Flow:
1. Wet Sense Unit triggers a wet event
2. Disables the wet sense interrupt.
3. Determine what the diaper resistance value is.
   a. Set resistance to 25 KOhm
   b. If tripped, set threshold value to one half less than current value recheck threshold.
      i. Continue until non trip threshold is found, comparator is no longer tripped.
      ii. Once found add % of the current value until comparator trips
      iii. Reduce by 1 KOhm steps until comparator is no longer tripped.
      iv. This is the current diaper resistance level.
4. Send Wet Event message to the system with the current diaper resistance level (50 KOhm for dry diaper).
   a. Message format defined below.
5. Allow diaper to absorb wet event. Sleep for 5 minutes
6. Determine what the diaper resistance value is.
   a. Set resistance to 25 KOhm
   b. If tripped, set threshold value to one half less than current value recheck threshold.
      i. Continue until non trip threshold is found, comparator is no longer tripped.
      ii. Once found add ¼ of the current value until comparator trips
      iii. Reduce by 1 KOhm steps until comparator is no longer tripped.
      iv. This is the current diaper resistance level.
7. Subtract 5 KOhm to set the new threshold resistance value.
8. If resistance threshold level is >10 KOhm enable edge detection mode, if <10 KOhms enable periodic level sense mode.

Wet Event Message Format:

In the current wet event message the 8 bit wet count field that is currently not used.

The current wet module message format is shown in the table below

TABLE
Old (WAS) Wet Message Format

| | Byte | | | | | | |
|---|---|---|---|---|---|---|---|
| | Byte 0 | Byte 1 | Byte 2-9 | Byte 10 | Byte 10-11 | Byte 12 | Byte 13-14 |
| Name | Wet | delimiter | Sensor | delimiter | Battery | delimiter | Count |
| | | | MAC ID | | Voltage | | |
| Value | Asci “W” | Asci ‘\hat{ }’ | 8xAsci | Asci ‘\hat{ }’ | ADCH(7:0) ++ | Asci ‘\hat{ }’ | Asci value |
| | | | Char | | ADCL(7:2) | | |

To transmit all the information required for the new dynamic sensor but to minimize message length the currently unused “Wet Counter” byte will be repurpose and add an additional byte will be added. The new message format definition will be defined below.

Note, adding a byte to the overall message length is not significant because all zigbee message payloads utilize delimiter “̂” between each field enabling dynamic message length functionality. So repurposing and extending the length of the Wet counter will only impact that part of the system processing and all other field processing will remain unchanged.

Below is the new wet event message format. The Wet Count byte has been replaced with a two byte field labeled “Wet State”

TABLE
New (IS) Wet Message Format

| | Byte | | | | | | |
|---|---|---|---|---|---|---|---|
| | Byte 0 | Byte 1 | Byte 2-9 | Byte 10 | Byte 10-11 | Byte 12 | Byte 13-16 |
| Name | Wet | delimiter | Sensor | delimiter | Battery | delimiter | Wet State |
| | | | MAC ID | | Voltage | | |
| Value | Asci “W” | Asci ‘\hat{ }’ | 8xAsci | Asci ‘\hat{ }’ | ADCH(7:0) ++ | Asci ‘\hat{ }’ | Resistance |
| | | | Char | | ADCL(7:2) | | Triggered and |
| | | | | | | | New Level |

The new message field “Wet State”, reports the current event type and pre and post wet resistance values.

The Wet State field is defined below.

TABLE
Wet State Definition

| | Bit | | | | | | | |
|---|---|---|---|---|---|---|---|---|
| | Bit 15 | Bit 14 | Bit 13 | Bit 12 | Bit 11 | Bit 10 | Bit 9 | Bit 8 |
| Name | X | X | X | X | X | X | X | X |

| Value | Triggered Wet Resistor Value: The resistance value the wet event triggered on, 50K-0 Ohm | | | | | | | |
|---|---|---|---|---|---|---|---|---|
| | Bit | | | | | | | |
| | Bit 7 | Bit 6 | Bit 5 | Bit 4 | Bit 3 | Bit 2 | Bit 1 | Bit 0 |
| Name | X | X | X | X | X | X | X | X |

| | |
|---|---|
| Value | New Wet Resistor Value: The new resistance value of the digital resistor after the wet event, 50K-0 Ohm. |
| | The new threshold value will we a resistance level TBD KOhm lower than this value. |

Triggered Wet Resistor Value: Is the digital potentiometer 8 bit register value that maps to a resistor value that the unit triggered on to generate the current Wet Event message.

New Wet Resistor Value: Is the new potentiometer 8 bit register value that the unit measured after the wet event. This value maps to the resistor value measured in the unit that does not result in the comparator to trip but is very close to this point.

Note the “New Resistor Value” Is NOT the new threshold value the sensor unit is set to sense subsequent events. The new Threshold value will be a count/resistance value TBD KOhms lower to create a bit of margin/hysteresis against detecting the next event and not false alarms on an already wet incontinent product.

8

Network NET Description

The Raspberry PI code will be updated to support the message decode of the new wet sense messages but still post the message to the local system website such that the system still works as designed today.

The code will be updated again once the system level web page has been updated to support the new message formats.

Detailed message formats are captured Monitoring and Wet Event sections above.

System Level Wet Sense Description

To support the new dynamic wet sense

The System level algorithm flow:
1. Incontinent users system profile is loaded, this profile informs the system what type of wetter this person is and how the system should respond to wet events from the users wet sensor unit, fields may include:
   a. Caregiver alert levels
   b. Hold Offs
   c. Persistence
   d. Resistance rate of change . . .
2. System waits for wet event message to arrive
3. Once new message arrives
   a. Message is logged
   b. Pre and post resistance values are analyzed
   c. Based on the analysis results and system settings for that specific incontinence user the system the following action is taken such as but not inclusive:
      i. Notify the caregiver the user is wet but does not need to be changed.
      ii. Notify the caregiver the user is wet and requires changing
      iii. Do not notify the caregiver but update system state saturation

Configuration

The configuration control of the AD5165 will be captured next.

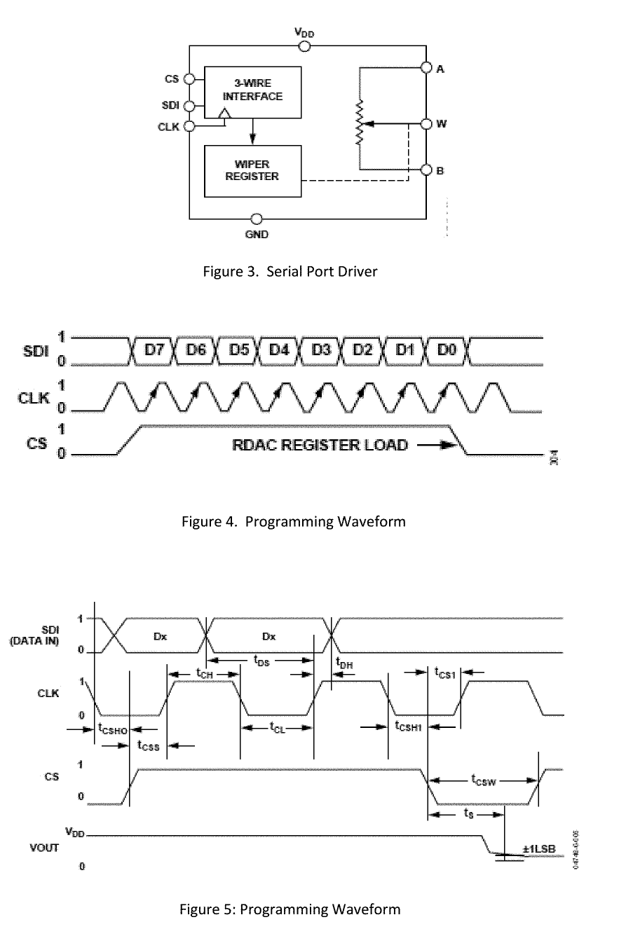

Configuring the W port requires the development of a serial port driver. Refer to FIGS. 3-6 and Table 1 & 2. for programming characteristics.

See FIG. 3. Serial Port Driver.

See FIG. 4. Programming Waveform

See FIG. 5. Programming Timing Guide

TABLE 1
Wetness Sensor Version 3 AD5165 connections

| AD5165 | CC2530 | | | |
|---|---|---|---|---|
| Pin | Pins | CC2530EM | Smart RF05EB | Function |
| VDD | P0_3 | P1:9 | P5:9 EM_UART_TX | Pwr On/Off Part |
| GND | GND | | | Ground |
| CS | P1_5 | P1:16 | P5:16 EM_SCLK | Chip Select, Enable to program |
| | | | | resistance |
| SDI | P1_4 | P1:14 | P5:14 EM_CS | Serial Data In, Load resistance |
| | | | | value on this line |
| CLK | P1_3 | P1:4 | P5:4 | Serial Data Clock, Toggle this line |
| | | | EM_FLASH_CS | to shift in value |
| A | P0_0 | P1:11 | P5:11 | Pull Up resistor I/O, pull high to |
| | | | EM_LCD_MODE | enable sensing |
| W | P0_5 | P2:18 | P6:18 | Diaper +, connects resistor to D+ |
| | | | EM_UART_RTS | and Comparator |
| B | NC | | | No connect |

TABLE 2
Timing Characteristics for Programming
TIMING CHARACTERISTICS -100 kΩ VERSION
Table 2.

| Parameter | Symbol | Condition | Min | Typ¹ | Max | Unit |
|---|---|---|---|---|---|---|
| 3-WIRE INTERFACE TIMING CHARACTERISTICS^{2,3,4}(specifications apply to all Parts) | | | | | | |
| Clock Frequency | f_{CLK} = 1/(t_{CH} + t_{CL}) | | | | 25 | MHz |
| Input Clock Pulse Width | t_{CH}, t_{CL} | Clock level high or low | 20 | | | ns |
| Data Setup Time | t_{DS} | | 5 | | | ns |
| Data Hold Time | t_{DH} | | 5 | | | ns |
| CS Setup Time | t_{CSS} | | 15 | | | ns |
| CS Low Pulse Width | t_{CSW} | | 40 | | | ns |
| CLK Fall to CS Rise Hold Time | t_{CSH0} | | 0 | | | ns |
| CLK Fall to CS Fall Hold Time | t_{CSH1} | | 0 | | | ns |
| CS Fall To Clock Rise Setup | t_{CS1} | | 10 | | | ns |

V_{DD} = +5 V ± 10%, or +3 V ± 10%; V_{A} = V_{DD}; V_{B} = 0 V; −40° C. < T_{A} < +125° C.; unless otherwise noted.

### SEQUENCE LISTING

Not Applicable

What is claimed:

1. A wetness sensing device which uses digital technology to manipulate an electronic circuitry reference point, allowing the circuit references to be changed by a software controlled algorithm.

2. A wetness sensing device as described in claim 1, wherein preprogrammed routines can be executed to allow the gathering of data that can then be analyzed not only for wetness but disease or other biometric findings.

3. A wetness sensing device as described in claim 1, which captures the sensing data and transmits it through a variety of means to a device which can then perform further analysis of the data and present logical conclusions and recommendations.

4. A wetness sensing device as described in claim 1, wherein the digital resistor enables increased dynamic range voltage measurement on a sensing circuit that can be read by several means through either a comparator circuit, an analog to digital measurement or any other circuit that can measure voltage or current either directly or indirectly.

5. A wetness sensing device as described in claim 1, with the implementation of a dynamic filter, based on a configurable subsampling temporal rate sensor input sampler and recording subsampled binary results that are accumulated and compared to threshold to determine final output state.

6. A wetness sensing device as described in claim 1, where the incontinence resistance, impedance, capacitance or conductance of the urine can be continuously monitored and measured, enabling the absolute and temporal difference measurement values be used to as predictive elements to either directly or indirectly identify the onset or presence of potential medical conditions.

7. A wetness sensing device as described in claim 6, where both an impedance and conductance measurement can be implemented to determine the saline content, to a much higher accuracy, which can be used to predict or identify the onset or presence of potential medical conditions.

8. A wetness sensing device as described in claim 1, wherein the sensing circuit will dynamically and in real time set the dynamic range and level of the sensor and in addition to setting the sense level, it will optimize the sensors sensing performance versus power performance and enable either a low power “always on” edge triggered sense mode or a temporal periodic level sense mode.
